(19) United States
(12) Patent Application Publication
(10) Pub. No.: US 2019/0328999 A1
(43) Pub. Date: Oct. 31, 2019

(54) GUIDEWIRE INTRODUCER AND STORAGE TUBE INTERFACE

(71) Applicant: Thomas A. SOS, New York, NY (US)

(72) Inventor: Thomas A. SOS, New York, NY (US)

(21) Appl. No.: 16/395,642

(22) Filed: Apr. 26, 2019

Related U.S. Application Data
(60) Provisional application No. 62/663,709, filed on Apr. 27, 2018.

(51) Int. Cl. A61M 25/00; A61M 25/09

(52) U.S. Cl. CPC A61M 25/002; A61M 25/09041

(57) ABSTRACT

A guidewire for medical procedures can be stored in a coiled configuration with a clamp. The clamp comprises a guidewire introducer funnel, which can be removably coupled to a hub of a catheter. The clamp comprises an adaptor to allow it to be removably coupled to a guidewire storage tube. The clamp comprises an interior channel configured to allow a guidewire to be advanced therethrough, and the interior channel can be coupled to the introducer and an adaptor that can be coupled to the back end of the clamp.

### CROSS-REFERENCE

This application claims priority to U.S. Patent Application No. 62/663,709 filed Apr. 27, 2018 [Attorney Docket No. 41145-706.101], the contents of which are incorporated herein by reference.

The subject matter of this application is related to the subject matter of the following patent applications: application Ser. No. 15/000,210 now U.S. Pat. No. 9,717,888 [Attorney Docket No. 41145-704.201]; and application Ser. No. 15/632,805 [Attorney Docket No. 41145-704.301] which are each incorporated herein by reference.

### BACKGROUND

The present disclosure relates to medical devices and methods, particularly for the storage of guidewires suitable for use in various medical procedures.

The following references may be of interest: U.S. Publication Nos. 2014/0259821, 2014/0171833, 2013/0292365, and 2012/0227751 and U.S. Pat. Nos. 8,679,065, 8,480,597, 7,886,906, 7,144,402, 6,047,825, 5,830,183, 5,279,573, 4,707,906, and D329698.

### SUMMARY

The present disclosure relates to medical devices and methods, particularly for the storage of guidewires suitable for use in various medical procedures.

In an aspect, a clamp for facilitating guidewire storage is provided. The clamp may comprise: a first jaw or portion, a second jaw or portion, and a pivotable portion therebetween; an interior channel in the first or second portion configured to allow a guidewire to be advanced therethrough; and a back-end or proximal adapter coupled to the interior channel and adapted to be removably coupled to a guidewire storage tube. The first and second portions may be foldable or pivotable relative to one another about the pivotable portion to open or close the clamp. The first and second portions may comprise inward-facing surfaces configured to approximate or contact one another when the clamp is closed. The inward-facing surfaces may have one or more surface features to facilitate capture of the guidewire between the inward-facing surfaces of the first and second portions.

In some embodiments, the clamp comprises one or more surface features which comprise a high friction material or coating. In some embodiments, the clamp comprises a high friction material or coating which is soft and compliant so as to minimize damage to a guidewire captured between the first and second portions.

In some embodiments, the clamp comprises a proximal adapter which is a male adapter sized and shaped to fit an interior of a guidewire storage tube. In some embodiments, the clamp comprises a proximal adapter which is a female adapter sized and shaped to fit an exterior of a guidewire storage tube.

In some embodiments, the clamp comprises one or more surface features which comprise teeth, ridges, or ribs to facilitate capture of one or more guidewire loops when the clamp is closed. In some embodiments, the clamp comprises a first portion which comprises a first set of teeth and a second portion which comprises a second set of teeth, the first and second sets being complementary to one another. In some embodiments, the clamp comprises a first and second set of teeth which fit within one another when the first and second portions are folded to close the clamp. In some embodiments, the clamp comprises tips of the first and second set of teeth which abut one another when the first and second portions are folded to close the clamp.

In some embodiments, the clamp comprises one or more teeth shaped to accommodate one or more guidewire loops which comprises a plurality of cut-outs arranged in series, each cut-out being configured to accommodate a single guidewire loop. In some embodiments, the clamp comprises one or more teeth shaped to accommodate the one or more guidewire loops which comprises a cut-out having a plurality of semi-circular sections, the cut-out being configured to accommodate a plurality of guidewire loops. In some embodiments, the clamp comprises a first end coupled to the first portion and a second end coupled to the second portion, the first and second ends being removably coupleable to one another. In some embodiments, the clamp comprises a first end which comprises one or more teeth to capture the second end when the first and second portions are folded or pivoted relative to one another to close the clamp.

In some embodiments, the clamp comprises a guidewire introducer coupled to the interior channel, wherein the guidewire introducer has a guidewire lumen open to the interior channel, an open proximal end to receive the guidewire and direct the guidewire into the guidewire lumen, and a distal funnel tip to couple with a proximal hub of a catheter. In some embodiments, the clamp comprises a guidewire introducer which further comprises a grasping mechanism to secure the guidewire received in the guidewire lumen. In some embodiments, the clamp comprises a guidewire introducer which is removably coupled to one or more of the first or second portions.

In another aspect, a clamp for facilitating guidewire storage is provided. The clamp may comprise: a first jaw or portion, a second jaw or portion, and a pivotable portion therebetween; an interior channel in the first or second portion configured to allow a guidewire to be advanced therethrough; and a proximal adapter coupled to the interior channel and adapted to be removably coupled to a guidewire storage tube. The first and second portions may be foldable or pivotable relative to one another about the pivotable portion to open or close the clamp. The first and second portions may comprise inward-facing surfaces configured to approximate or contact one another when the clamp is closed, the inward-facing surfaces having one or more surface features to facilitate capture of the guidewire between the inward-facing surfaces of the first and second portions. The one or more surface features may comprise teeth, ridges, or ribs to facilitate capture of one or more guidewire loops when the clamp is closed. One or more of the first or second portions may comprise one or more teeth. The one or more teeth may be shaped to accommodate one or more guidewire loops. The one or more teeth shaped to accommodate the one or more guidewire loops may comprise a cut-out having a plurality of semi-circular sections. The cut-out may be configured to accommodate a plurality of guidewire loops, and wherein the clamp further comprises a soft membrane over at least a portion of the cut-out.

In some embodiments, the clamp comprises a first portion which comprises a first set of teeth and a second portion which comprises a second set of teeth, the first and second sets being complementary to one another. In some embodiments, the clamp comprises a first and second set of teeth which fit within one another when the first and second portions are folded to close the clamp. In some embodiments, the clamp comprises tips of the first and second set of teeth which abut one another when the first and second portions are folded to close the clamp.

In some embodiments, the clamp comprises a back-end or proximal adapter which is a male adapter sized and shaped to fit an interior of a guidewire storage tube. In some embodiments, the clamp comprises a proximal adapter which is a female adaptor sized and shaped to fit an exterior of a guidewire storage tube.

In some embodiments, the clamp comprises a portion of the one or more teeth shaped to accommodate one or more guidewire loops which comprises a plurality of cut-outs arranged in series, each cut-out being configured to accommodate a single guidewire loop. In some embodiments, the clamp comprises a soft membrane which comprises a high friction material or coating. In some embodiments, the clamp comprises a high friction material or coating which is soft and compliant so as to minimize damage to a guidewire captured between the first and second portions.

In some embodiments, the clamp comprises a first end coupled to the first portion and a second end coupled to the second portion, the first and second ends being removably coupleable to one another. In some embodiments, the clamp comprises a first end which comprises one or more teeth to capture the second end when the first and second portions are folded or pivoted relative to one another to close the clamp. In some embodiments, the clamp comprises a guidewire introducer coupled to the interior channel, wherein the guidewire introducer has a guidewire lumen open to the interior channel, an open proximal end to receive the guidewire and direct the guidewire into the guidewire lumen, and a distal funnel tip to couple with a proximal hub of a catheter. In some embodiments, the clamp comprises a guidewire introducer which further comprises a grasping mechanism to secure the guidewire received in the guidewire lumen. In some embodiments, the clamp comprises a guidewire introducer which is removably coupled to one or more of the first or second portions.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a section view of a guidewire clamp with a female adapter which may be coupled to the end of a guidewire storage tube, in accordance with some embodiments.

FIG. 2 shows a section view of a guidewire clamp with a male adapter which may be coupled to the end of a guidewire storage tube, in accordance with some embodiments.

FIG. 3 shows a section view of a guidewire clamp comprising an alternative hinge mechanism, in accordance with some embodiments.

FIG. 4 shows an isomorphic view of a guidewire clamp, in accordance with some embodiments.

FIG. 5 shows an isomorphic view of the guidewire clamp with an alternative hinge design, in accordance with some embodiments.

FIG. 6 shows an image of a guidewire clamp decoupled from a guidewire in a closed position, in accordance with some embodiments.

FIG. 7 shows an image of a guidewire clamp engaging a guidewire, in accordance with some embodiments.

FIG. 8 shows an image of a guidewire clamp comprising a guidewire within the introducer portion and comprising a storage tube coupled to the clamp, in accordance with some embodiments.

FIG. 9 shows an image a detail view of the guidewire clamp of FIG. 8, in accordance with some embodiments.

### DETAILED DESCRIPTION OF THE INVENTION

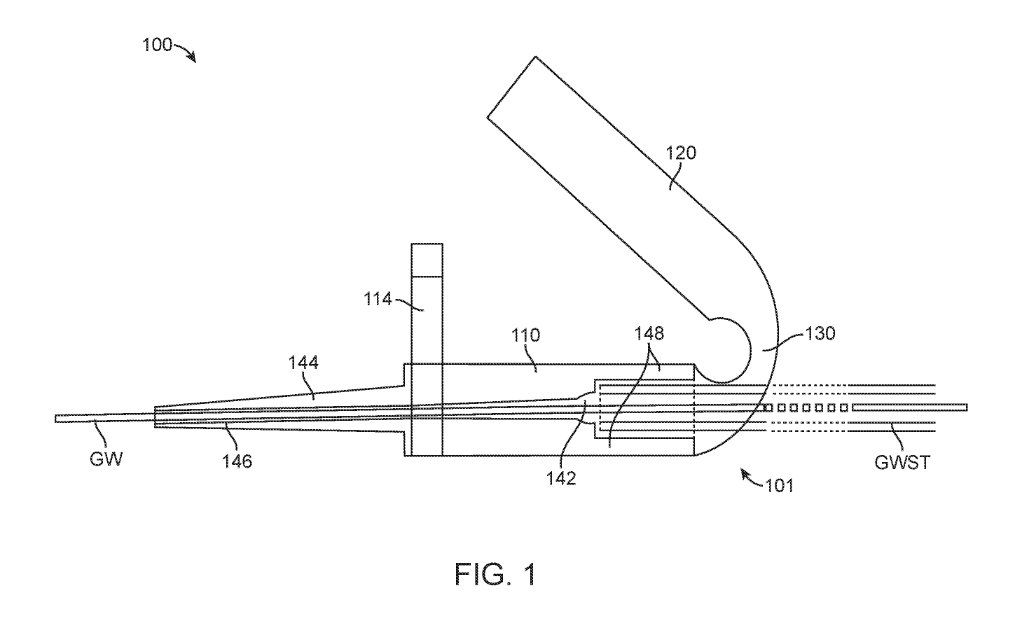

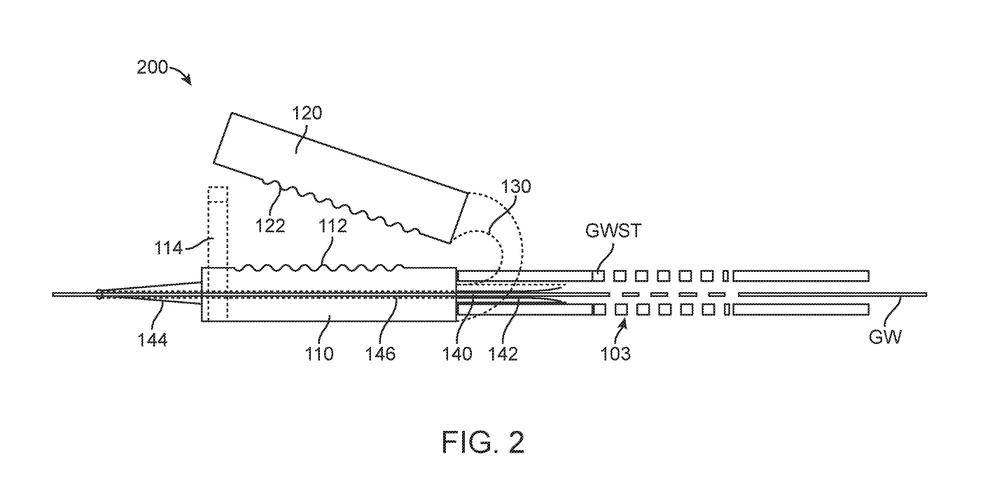

FIGS. 1 and 2 shows section views of guidewire clamps 100, 200, respectively, which may be coupled to the end of a guidewire storage tube, in accordance with some embodiments. FIG. 1 shows a guidewire clamp 100 with a female adapter 101 and FIG. 2 shows a guidewire clamp 200 with a male adapter 102. The guidewire clamps 100, 200 may comprise a guidewire lumen 146, through which a guidewire GW may be slidably inserted. The clamp 100, 200 may comprise an upper jaw or portion 120 and a lower jaw or portion 110. The upper portion 120 and the lower portion 110 may be connected by a hinge or spring 130. The upper portion 120 and lower portion 110 may be foldable or pivotable to open or close the clamp 110. When the clamp 100, 200 are closed, the upper portion 120 and the lower portion 110 may have adjacent surfaces, which may be in contact or approximately in contact in the closed position.

The upper portion 120 and/or the lower portion 110 may comprise surface features 122, 112, respectively, typically present on the interiorly facing side or surface of each of the upper or lower jaws. The surface features 122, 112 may comprise a compressible coating which conforms to fit a guidewire positioned inside the closed surfaces. The surface features 122, 112 may comprise teeth, ridges, or ribs to facilitate capture of one or more guidewire loops when the clamp is closed. The surface(s) may comprise a coating which prevents slippage of one or more guidewire loops in the interior of the closed clamp without damage to the one or more guidewire loops clamped therein. The clamps 100, 200 may each comprise one or more sets of teeth 122, 112 which may be complementary to one another. Additionally or alternatively, the sets of teeth 122, 112 may fit within each other. The teeth 122, 112 may be shaped to accommodate one or more guidewire loops contained therein. For example, the teeth 122, 112 may comprise cut-outs arrange in series, each cut-out being configured to accommodate one or more guidewire loops.

The clamps 100, 200 may comprise a clip or lock 114 to secure the upper portion 120 and the lower portions 110 in a closed position. The clip or lock 114 may removably couple a portion of the upper jaw 120 and the lower jaw 110 to one another. In some embodiments, the lock or clip 114 couples distal portions of the upper portion 120 and the lower portion 110 to one another. Further surface features and/or clipping or locking elements are described in the following patent applications: application Ser. No. 15/000,210 now U.S. Pat. No. 9,717,888 [Attorney Docket No. 41145-704.201]; and application Ser. No. 15/632,805 [Attorney Docket No. 41145-704.301] which are each incorporated herein by reference.

The distal end of the guidewire clamps 100, 200 may comprise a tapered front or distal end 144. The tapered end 144 may come to a tip, which may be introduced into a catheter hub. The tapered end 144 may comprise a guidewire introducer. The interior channel or lumen 146 of the guidewire introducer may be longitudinally aligned with the jaw the introducer is coupled to. The guidewire introducer may be a part of the body of the upper jaw 120 or the lower jaw 110, and the interior channel or lumen 146 may traverse the length of the upper jaw 120 or the lower jaw 110. The guidewire introducer may have a proximal end opposite the tapered distal end 144. Referring back to the tapered end 144, the introducer may comprise a distal funnel tip to introduce a guidewire GW into the lumen of a catheter hub. The tip 144 of the guidewire clamp 100 may comprise a grasping mechanism to secure the guidewire GW received within the lumen 146 of the distal tip 144. The lumen 146 of the funnel structure 144 may comprise a larger diameter on a distal end than a proximal end. Optionally, the diameter of the proximal end may comprise a similar diameter to the interior diameter of the lumen of the guidewire clamp.

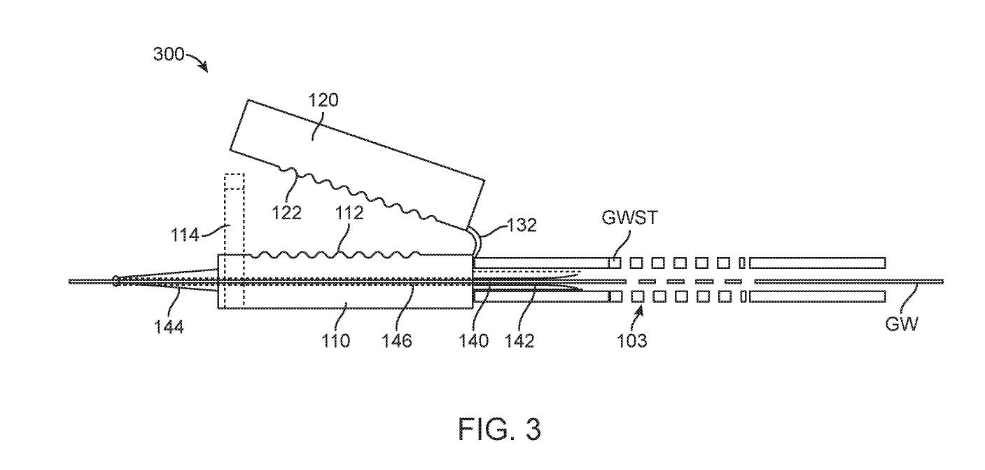

The guidewire introducer may comprise an adapter configured to be coupled to a guidewire storage tube GWST. The adapter may comprise a male adapter 103 (as shown in FIGS. 2 and 3) protruding from the proximal end of the jaw to interface with the interior channel or lumen of the guidewire storage tube GWST. The male adaptor 103 may comprise a proximal process as described herein and in the following paragraphs. Alternatively, the adapter may comprise a female adaptor 101 (as shown in FIG. 1), for example, positioned within the jaw, and adapted to interface with the outer body and end of the guidewire storage tube GWST. The female adaptor 101 may comprise the proximal lumen described herein and with respect to FIGS. 1, 8, and 9. The adaptor may comprise an interior funnel 142 to guide a guidewire from the guidewire storage tube into the interior channel or lumen of the adaptor and then the introducer.

In some embodiments, the clamps 100, 200 may comprise a process 140 on a proximal end of the clamps 100, 200. The cylindrical process 140 may comprise a lumen 142 through which a guide wire GW can be inserted. The lumen 142 may comprise a funnel structure interior to the lumen to guide the guidewire GW into the lumen 146 of the clamp 100. The exterior of the process 140 may be sized and shaped to fit inside of the guidewire storage tube GWST. The exterior of the process 140 may be sized and shaped to slightly stretch the interior of the guidewire storage tube GWST, such that the guidewire storage tube GWST is removably coupled to the clamp 100. When in use, the process 140 may aid in inserting a guidewire GW into the hub of a catheter without damage to the guidewire GW, such as crimping.

In the illustrated embodiment, the hinge portion 130 may be substantially external to the upper and lower portions. In some embodiments, the hinge portion contacts a back-end or proximal portion of the upper portion 120 and the lower portion 110 of the guidewire clamp 100. In some embodiments, the hinge mechanism 130 may be larger and to the side of the proximal adaptor. In some embodiments, the hinge portion 130 is under tension such that when a clasp or lock is opened the hinge springs open.

FIG. 3 shows a schematic of a guidewire clamp 300 comprising an alternative hinge mechanism 132, in accordance with some embodiments. In the illustrated embodiment, the hinge portion 132 connects the inside ends of the jaws 120, 110 of the clamp 300. Apart from the hinge mechanism 132, the embodiment of FIG. 3 has substantially similar features to the hinges 100 and 200 of FIGS. 1-2 including the proximal process 140. In some embodiments, a guidewire clamp with a female adapter 110 similar to that of FIG. 1 may utilize this alternative hinge mechanism 132.

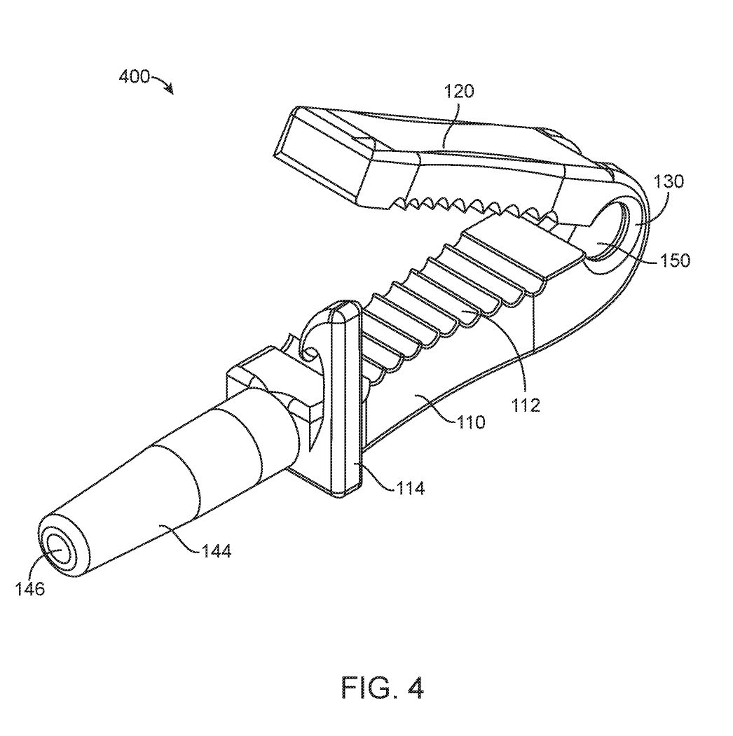

FIG. 4 shows an isomorphic view of a guidewire clamp 400, in accordance with some embodiments. The clamp 400 may include many features and elements in common with the clamps 100, 200, and 300. The clamp 400 is shown in an open position and with a spring hinge 130 connected to the sides of the proximal end of the upper portion 120 and the lower portion 110. The clamp 400 of FIG. 4 may not have a proximal process 140. However, in alternative embodiments, the clamp 400 of FIG. 4 may have a proximal process 140 as described with respect to FIG. 2 and FIG. 3. In embodiments where the clamp 400 does not have a proximal process, the proximal end may comprise a lumen 150 with a larger diameter than lumen 146 within the distal end 144 of the guide wire clamp. The lumen of the proximal end 150 may be sized and shaped to fit the guidewire storage tube GWST within the proximal lumen of the clamp 100. The lumen of the proximal end 150 may be sized and shaped such that the guidewire storage tube GWST is slightly compressed, such that the distal end of the guidewire storage tube GWST is removably coupled to the clamp 100.

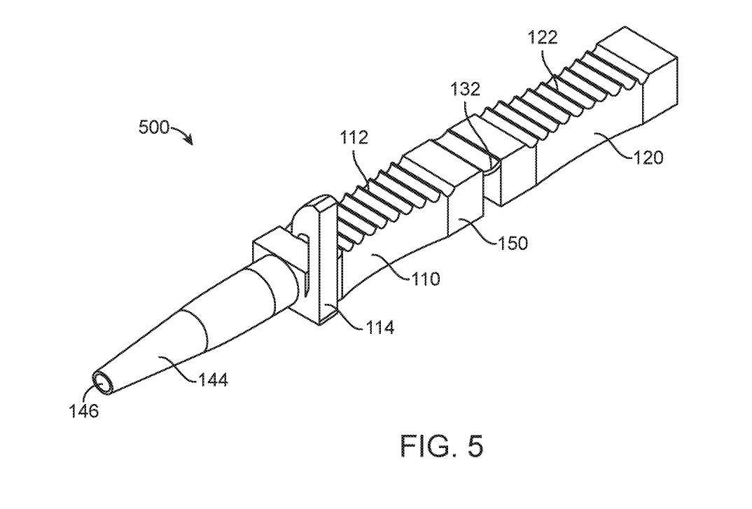

FIG. 5 shows an isomorphic view of the guidewire clamp 500 with an alternative hinge design 132, in accordance with some embodiments. The clamp 500 may include many features and elements in common with the clamps 100, 200, 300, and 400. The clamp 500 is shown in an open position. In the illustrated embodiment, the hinge portion 132 connects the inside ends of the jaws 120, 110 of the clamp 500. The clamp of FIG. 5 may not have a proximal process. However, in alternative embodiments, the clamp 500 of FIG. 5 may have a proximal process as described with respect to FIG. 2 and FIG. 3.

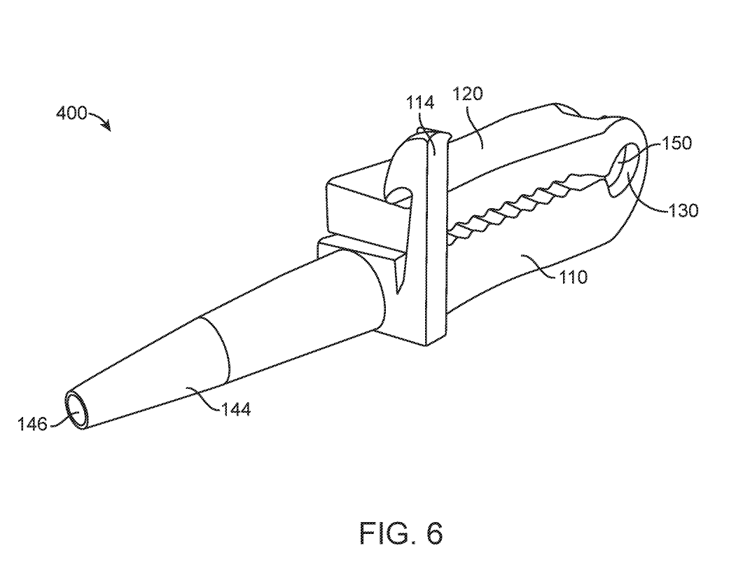

FIG. 6 shows an image of the guidewire clamp 400 decoupled from a guidewire GW in a closed position, in accordance with some embodiments. The guide wire clamp 400 may be of a substantially similar design to the clamp 400 of FIG. 4.

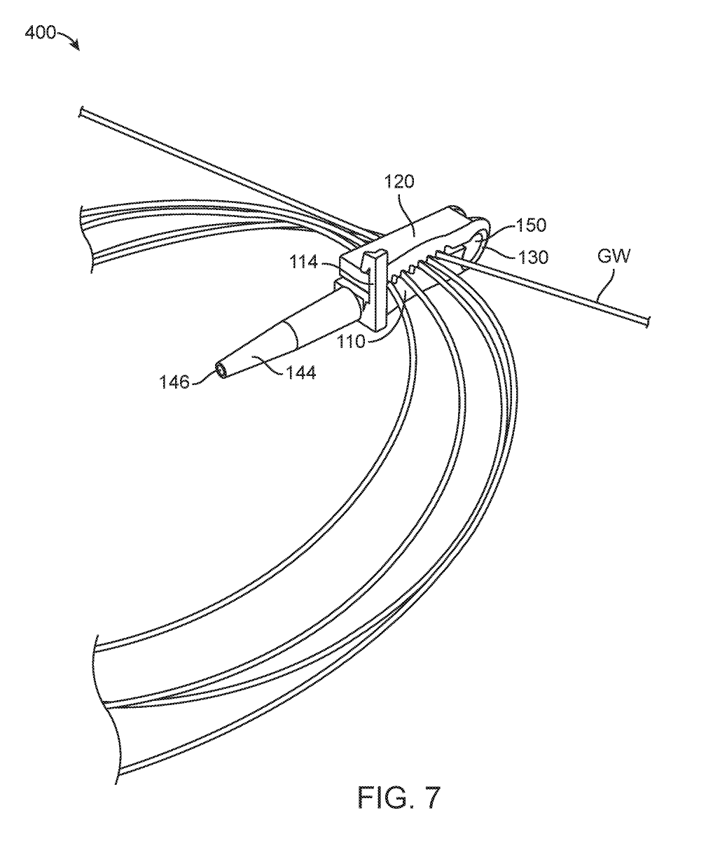

FIG. 7 shows an image of a guidewire clamp 400 engaging a guidewire GW, in accordance with some embodiments. In some embodiments, the guidewire clamp 400 facilitates guidewires storage by holding a guidewire GW within the upper and lower portions 120, 110 of the clamp 400. In some embodiments, the clamp 400 may be stored in a fluid filled bowl. The clamp 400 may be stored with other guidewire clamps 100, 200, 300, and/or 400 in a fluid filled bowl. The clamp 400 may be labeled or comprise labeling portions. A label may comprise a color or a colored portion. Additionally or alternatively, the label may comprise writing or a portion with writing thereon. The body of the clamp 100 may comprise a transparent or semi-transparent material such that at least a part of the clamped guidewire may be visible through the material of the jaw(s). The other guidewire clamps 100, 200, 300, and/or 400 may be similarly labeled or comprise labeling portions as well.

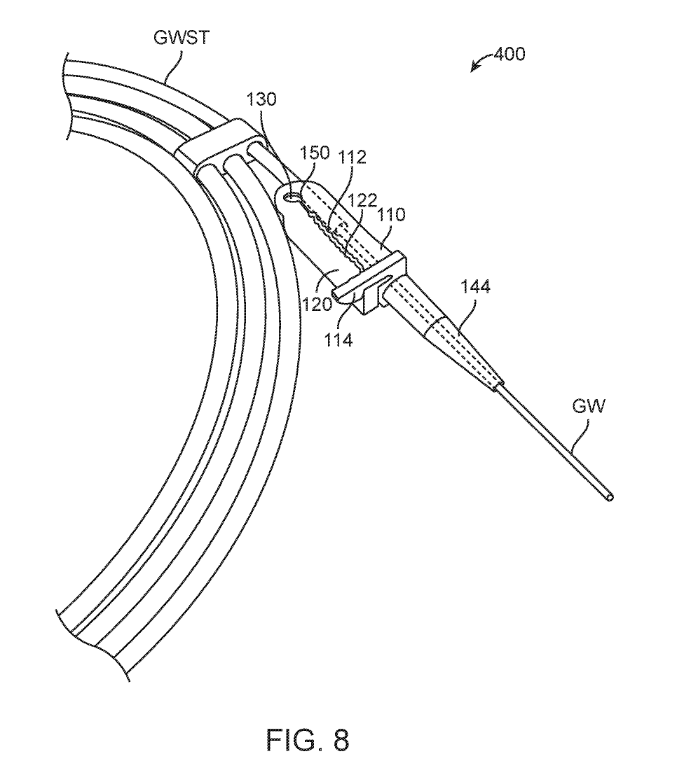

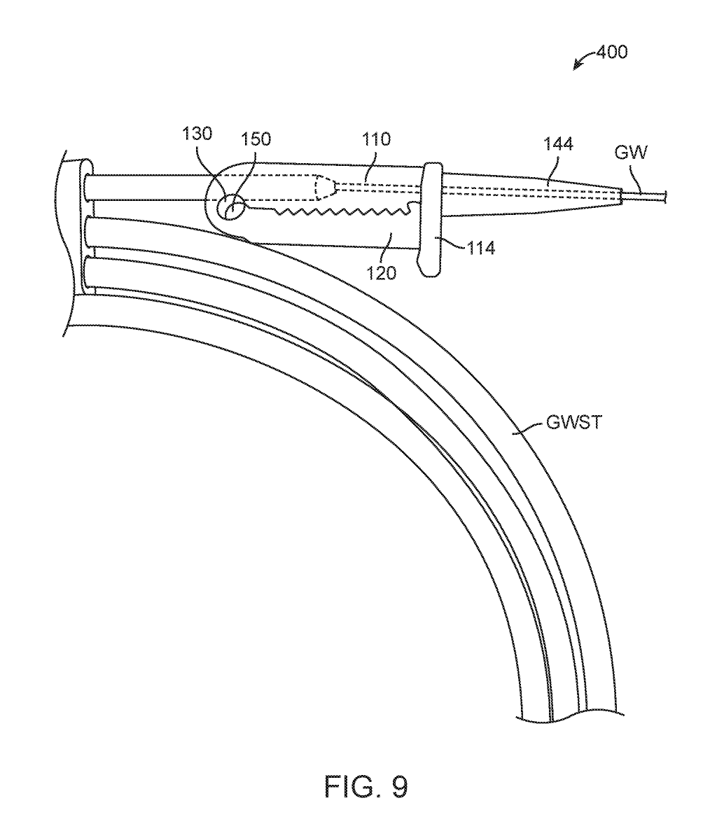

FIG. 8 shows an image of a guidewire clamp 400 having a female adapter 101 having a guidewire GW within the introducer portion and comprising a storage tube coupled to the clamp 100, in accordance with some embodiments. FIG. 9 shows an image a detail view of the guidewire clamp 400 of FIG. 8, in accordance with some embodiments. As illustrated, a guidewire GW may be slidably inserted into the lumen 146 of the guidewire clamp 400. The clamp 100 may be open or closed during use. In the illustrated embodiment, the clamp has a proximal process, and the proximal end may comprise a lumen 150 with a larger diameter than lumen within the distal end 146 of the guidewire clamp 400. The lumen of the proximal end 150 may be sized and shaped to fit the guidewire storage tube GWST within the proximal lumen of the clamp 150 and can function as an adapter. The lumen of the proximal end 150 may be sized and shaped such that the guidewire storage tube GWST is slightly compressed, such that the distal end of the guidewire storage tube is removably coupled to the clamp.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A clamp for facilitating guidewire storage, the clamp comprising:
a first portion, a second portion, and a pivotable portion therebetween;
an interior channel in the first or second portion configured to allow a guidewire to be advanced therethrough; and
a proximal adapter coupled to the interior channel and adapted to be removably coupled to a guidewire storage tube,
wherein the first and second portions are foldable or pivotable relative to one another about the pivotable portion to open or close the clamp, and
wherein the first and second portions comprise inward-facing surfaces configured to approximate or contact one another when the clamp is closed, the upper surfaces having one or more surface features to facilitate capture of the guidewire between the upper surfaces of the first and second portions.

2. The clamp of claim 1, wherein the one or more surface features comprise a high friction material or coating.

3. The clamp of claim 2, wherein the high friction material or coating is soft and compliant so as to minimize damage to a guidewire captured between the first and second portions.

4. The clamp of claim 1, wherein the proximal adapter is a male adapter sized and shaped to fit an interior of a guidewire storage tube.

5. The clamp of claim 1, wherein the proximal adapter is a female adapter sized and shaped to fit an exterior of a guidewire storage tube.

6. The clamp of claim 1, wherein the one or more surface features comprise teeth, ridges, or ribs to facilitate capture of one or more guidewire loops when the clamp is closed

7. The clamp of claim 1, wherein the first portion comprises a first set of teeth and the second portion comprises a second set of teeth, the first and second sets being complementary to one another.

8. The clamp of claim 7, wherein the first and second set of teeth fit within one another when the first and second portions are folded to close the clamp.

9. The clamp of claim 7, wherein tips of the first and second set of teeth abut one another when the first and second portions are folded to close the clamp.

10. The claim of claim 1, wherein the one or more teeth shaped to accommodate one or more guidewire loops comprises a plurality of cut-outs arranged in series, each cut-out being configured to accommodate a single guidewire loop.

11. The clamp of claim 1, wherein the one or more teeth shaped to accommodate the one or more guidewire loops comprises a cut-out having a plurality of semi-circular sections, the cut-out being configured to accommodate a plurality of guidewire loops.

12. The clamp of claim 1, further comprising a first end coupled to the first portion and a second end coupled to the second portion, the first and second ends being removably coupleable to one another.

13. The clamp of claim 12, wherein the first end comprises one or more teeth to capture the second end when the first and second portions are folded or pivoted relative to one another to close the clamp.

14. The clamp of claim 1, further comprising a guidewire introducer coupled to the interior channel, wherein the guidewire introducer has a guidewire lumen open to the interior channel, an open proximal end to receive the guidewire and direct the guidewire into the guidewire lumen, and a distal funnel tip to couple with a proximal hub of a catheter.

15. The clamp of claim 14, wherein the guidewire introducer further comprises a grasping mechanism to secure the guidewire received in the guidewire lumen.

16. The clamp of claim 14, wherein the guidewire introducer is removably coupled to one or more of the first or second portions.

17. A clamp for facilitating guidewire storage, the clamp comprising:
a first portion, a second portion, and a pivotable portion therebetween;
an interior channel in the first or second portion configured to allow a guidewire to be advanced therethrough; and
a proximal adapter coupled to the interior channel and adapted to be removably coupled to a guidewire storage tube,
wherein the first and second portions are foldable or pivotable relative to one another about the pivotable portion to open or close the clamp,
wherein the first and second portions comprise inward-facing surfaces configured to approximate or contact one another when the clamp is closed, the inward-facing surfaces having one or more surface features to facilitate capture of the guidewire between the inward-facing surfaces of the first and second portions,
wherein the one or more surface features comprise teeth, ridges, or ribs to facilitate capture of one or more guidewire loops when the clamp is closed,
wherein one or more of the first or second portions comprise one or more teeth,
wherein the one or more teeth are shaped to accommodate one or more guidewire loops,
wherein the one or more teeth shaped to accommodate the one or more guidewire loops comprises a cut-out having a plurality of semi-circular sections, the cut-out being configured to accommodate a plurality of guidewire loops, and
wherein the clamp further comprises a soft membrane over at least a portion of the cut-out.

18. The clamp of claim 17, wherein the first portion comprises a first set of teeth and the second portion comprises a second set of teeth, the first and second sets being complementary to one another.

19. The clamp of claim 18, wherein the first and second set of teeth fit within one another when the first and second portions are folded to close the clamp.

20. The clamp of claim 18, wherein tips of the first and second set of teeth abut one another when the first and second portions are folded to close the clamp.

21. The clamp of claim 17, wherein the proximal adapter is a male adapter sized and shaped to fit an interior of a guidewire storage tube.

22. The clamp of claim 17, wherein the proximal adapter is a female adaptor sized and shaped to fit an exterior of a guidewire storage tube.

23. The clamp of claim 17, wherein a portion of the one or more teeth shaped to accommodate one or more guidewire loops comprises a plurality of cut-outs arranged in series, each cut-out being configured to accommodate a single guidewire loop.

24. The clamp of claim 17, wherein the soft membrane comprises a high friction material or coating.

25. The clamp of claim 17, wherein the high friction material or coating is soft and compliant so as to minimize damage to a guidewire captured between the first and second portions.

26. The clamp of claim 17, further comprising a first end coupled to the first portion and a second end coupled to the second portion, the first and second ends being removably coupleable to one another.

27. The clamp of claim 26, wherein the first end comprises one or more teeth to capture the second end when the first and second portions are folded or pivoted relative to one another to close the clamp.

28. The clamp of claim 17, further comprising a guidewire introducer coupled to the interior channel, wherein the guidewire introducer has a guidewire lumen open to the interior channel, an open proximal end to receive the guidewire and direct the guidewire into the guidewire lumen, and a distal funnel tip to couple with a proximal hub of a catheter.

29. The clamp of claim 28, wherein the guidewire introducer further comprises a grasping mechanism to secure the guidewire received in the guidewire lumen.

30. The clamp of claim 28, wherein the guidewire introducer is removably coupled to one or more of the first or second portions.
